(19) United States
(12) Patent Application Publication
(10) Pub. No.: US 2026/0114896 A1
(43) Pub. Date: Apr. 30, 2026

(54) ADAPTIVE ROBOTIC SYSTEM AND METHOD FOR NEEDLE GUIDANCE

(71) Applicant: Georgia Tech Research Corporation, Atlanta, GA (US)

(72) Inventors: Yue Chen, Atlanta, GA (US); Samuel Wilcox, Atlanta, GA (US); Yilin Cai, Atlanta, GA (US)

(21) Appl. No.: 19/376,128

(22) Filed: Oct. 31, 2025

Related U.S. Application Data
(60) Provisional application No. 63/714,808, filed on Oct. 31, 2024.

(51) Int. Cl. A61B 17/34

(52) U.S. Cl. CPC A61B 17/3403; A61B 2017/3407

(57) ABSTRACT

An exemplary system and method for a flexible XY-stage that spans a substantial portion of a subject torso to assist in guiding a needle template or needle insertion apparatus for needle-based intervention procedures in an MRI scanner. The flexible XY-stage is optimally configured to span the entire torso of the subject in providing both (i) a large bendable frame that can be positioned over the subject and (ii) a low height profile, to optimally fit within the bore of an MRI scanner. The needle template or needle insertion apparatus is movable within a large movable space defined by the flexible XY-stage and guided by cables connected at corners of the flexible XY-stage. The needle template or needle insertion apparatus has a plurality of mounting positions that allows a needle-based instrument to be mounted to the needle template or needle insertion apparatus and over a large degree of angle.

### RELATED APPLICATION

This U.S. application claims priority to, and the benefit of, U.S. Provisional Patent Application No. 63/714,808, filed Oct. 31, 2024, entitled “ADAPTIVE ROBOTIC SYSTEM AND METHOD FOR NEEDLE GUIDANCE,” which is incorporated by reference herein in its entirety.

### BACKGROUND

Certain medical treatments (ablation, chemotherapy) or diagnoses (biopsies) require the placement of a needle into an organ, tissue, or interstitial spaces within a patient. For example, clinicians would procedurally insert a radiofrequency ablation (RFA) needle into a patient's organ, e.g., for cancer treatment and biopsy. Even with intraprocedural image guidance, the procedure can be technically challenging due to respiration-induced movements of the organ(s). Needle targeting error has been observed to be as large as 5.5 cm in the superior-inferior direction.

Certain medical procedures and treatments, e.g., for the treatment of certain cancers, can only be treated or observed through magnetic resonance imaging (MRI) scans. Certain tumors cannot be differentiated and visualized only under MRI. While open bore MRI systems are available, they are equipped with lower field strength magnets that limit the resolution and quality of their scans.

Microwave ablation (MWA), for example, has proven effective in treating hepatocellular carcinoma (HCC) or small-sized liver tumors, but the state-of-the-art technique experiences suboptimal workflow due to the limited accuracy provided by manual needle insertions. Both MWA and other needle-based interventions require precise and safe needle trajectories toward dynamically moving targets (e.g., within the liver or other organs such pancreas or kidney). In the current practice, clinicians increase the ablation power to generate a larger ablation zone to compensate for the inaccurate needle placement, but this will inevitably lead to unintended collateral thermal injury, particularly when they are located near critical structures such as the colon, diaphragm, gallbladder, or main bile duct.

There is a benefit to having improved robotic systems for needle delivery and other minimally invasive needle-based intervention procedures in MRI scanners.

### SUMMARY

An exemplary system and method are disclosed for a flexible XY-stage that spans a substantial portion of a subject's torso to assist in guiding a needle template or needle insertion apparatus for needle-based intervention procedures in an MRI scanner. The flexible XY-stage is optimally configured to span the entire torso of the subject in providing both (i) a large bendable frame that can be positioned over the subject and (ii) a low height profile, to optimally fit within the bore of an MRI scanner. The needle template or needle insertion apparatus is movable within a large movable space defined by the flexible XY-stage and guided by cables connected at the corners of the flexible XY-stage. The needle template or needle insertion apparatus has a plurality of mounting positions (e.g., rings) that allows a needle-based instrument to be mounted to the needle template or needle insertion apparatus and over a large degree of angle, notably 0° to 75° from vertical, allowing for more options for a clinician to place the needle-based instrument in the patient, e.g., to reach an abdomen target without damaging the obstacles (e.g., blood vessels).

The flexible XY-stage may be optimized by discretizing the curved surface into multiple flat surfaces and applying a planar kinematics model to solve for the robot kinematics. The exemplary system and method can provide precise position and orientation control of the needle because its domed needle template can, via customizable holes (i.e., rings), allow the needles to reach an abdomen target without damaging the obstacles (e.g., blood vessels). The exemplary system and method can notably conform to the patient's contours and maintain structure integrity. The flexible XY-stage can adapt to a broad range of patient types, body weight indices, and orientations. In some embodiments, the flexible XY-stage can incorporate a frame made at least in part of deformable materials (e.g., thermoplastic polyurethane (TPU)) or rigid materials with a flexible structure design. In some embodiments, the XY-stage can employ motors to drive the needle guide at various locations and orientations. The XY-stage can also be controlled manually. While beneficial for a close-bore MRI scanner, the exemplary system and method can be employed for open-bore MRI, wide-bore MRI, and other MRI systems.

In an aspect, an apparatus is disclosed comprising a first frame arm and a second frame arm connected by a set of flexible arms (e.g., flexible beam or a set of interconnected members) to form an open flexible frame configured to bend (e.g., along a single axis) around a body region (e.g., torso, pelvic, or lower extremity region) of a subject, the open flexible frame defining a movable space for a guiding component; and a movable assembly as the guiding component tethered, via a cable, to each corner of the first arm and the second arm, the open flexible frame maintaining the cable in tension to guide movement of the movable assembly in the movable space, the movable assembly including a guide or motorized component to fixably maintain a needle instrument for needle placement.

In some embodiments, the open flexible frame defines the movable space for a substantial portion of a torso region as the body region of the subject (e.g., greater than 50% of torso area).

In some embodiments, the open flexible frame spans across a plane when in an unbending configuration, wherein the movable assembly is positionable and movable in the plane to provide a low-profile height device.

In some embodiments, the open flexible frame is configured to span a substantial portion of an MRI bore (e.g., at least 50% along a largest plane defined in the bore).

In some embodiments, the movable assembly (e.g., domed template) includes as a substrate having a plurality of guiding holes for placement of the needle instrument, including a first guiding hole and a second guiding hole, wherein a first guiding hole provides a first orientation of the needle instrument, and wherein a second guiding hole provides a second orientation of the needle instrument, the guiding holes providing an insertion angles range of at least 60 degrees (e.g., 0° to 75° from vertical or normal of the body surface).

In some embodiments, the movable assembly includes a substrate having a plurality of guiding holes for placement of the needle instrument.

In some embodiments, the open flexible frame has a symmetric rectangular or square profile.

In some embodiments, the open flexible frame has an asymmetric rectangular or square profile.

In some embodiments, the open flexible frame is configured to conform to a patient's contours at the body region.

In some embodiments, at least the set of interconnected members is made of a deformable material (e.g., thermoplastic polyurethane).

In some embodiments, the first frame arm and a second frame arm comprise a drive assembly comprising at least one bearing ring to guide the cable operatively couple thereto, the drive assembly (e.g., a ratchet and pawl subassembly) configured to provide resistance to or anchoring of the movement of the cable to maintain the movable assembly at a position.

In some embodiments, the drive assembly includes at least one actuator fixably coupled to the at least one bearing ring, the actuator being configured to drive the at least one bearing ring to reduce friction between the movable assembly and the patient's skin. [motorized]

In some embodiments, the movable assembly has a half-sphere profile.

In some embodiments, the first frame arm and a second frame arm include straps.

In some embodiments, the drive assembly comprises a ratchet and pawl subassembly to provide resistance to or anchoring of the movement of the cable.

In some embodiments, two or more cables are employed to traverse across the four corners of the open flexible frame to connect to the movable assembly.

In some embodiments, a single cable is employed to traverse across the four corners of the open flexible frame to connect to the movable assembly.

In another aspect, a method is disclosed comprising securely positioning (e.g., via straps) a needle guiding apparatus across a body region of a subject, wherein the needle guiding apparatus comprises: (i) a first frame arm and a second frame arm connected by a set of interconnected members to form an open flexible frame configured to bend (e.g., along a single axis) around a body region (e.g., torso, pelvic, or lower extremity region) of a subject, the open flexible frame defining a movable space for a guiding component; and (ii) a movable assembly as the guiding component tethered, via a cable, to each corner of the first arm and the second arm, the open flexible frame maintaining the cable in tension to guide movement of the movable assembly in the movable space, the movable assembly including a guide or motorized component to fixably maintain a needle instrument for needle placement. The method further includes scanning the subject with the needle guiding apparatus secured to the subject while in the MRI bore; positioning the movable assembly at a position of interest within the movable space for a needle placement; and rescanning and repositioning the needle instrument using the needle guiding apparatus to fixably maintain the needle instrument across the bore.

In another aspect, a method is disclosed comprising: securely positioning (e.g., via straps) a needle guiding apparatus across a body region of a subject, wherein the needle guiding apparatus comprises: (i) a first frame arm and a second frame arm connected by a set of interconnected members to form an open flexible frame configured to bend (e.g., along a single axis) around a body region (e.g., torso, pelvic, or lower extremity region) of a subject, the open flexible frame defining a movable space for a guiding component; and (ii) a movable assembly as the guiding component tethered, via a cable, to each corner of the first arm and the second arm, the open flexible frame maintaining the cable in tension to guide movement of the movable assembly in the movable space, the movable assembly including a guide or motorized component to fixably maintain a needle instrument for needle placement, wherein the movable assembly and/or the open flexible frame is motorized. The method further includes scanning the subject with the needle guiding apparatus secured to the subject while in the MRI bore; and positioning and repositioning the movable assembly at a position of interest within the movable space for a needle placement during an MRI scan.

In some embodiments, the method includes inserting, via the motorized movable assembly, the needle instrument into the subject while the subject is being scanned.

### BRIEF DESCRIPTION OF THE DRAWINGS

FIGS. 1A and 1B each show an example XY-Stage and needle guidance template configured to operate with an MRI system for needle guidance and/or placement in accordance with an illustrative embodiment.

FIGS. 2A and 2B each shows the flexible and expandable XY-Stage Frame for securely positioning positioned on a subject in accordance with an illustrative embodiment.

FIGS. 3A-3E show a manually operatable XY-Stage and needle guidance template of FIGS. 1A and 1B in accordance with an illustrative embodiment.

FIGS. 4A-4E show a motorized XY-Stage and needle guidance template of FIGS. 1A and 1B in accordance with an illustrative embodiment.

FIGS. 5A, 5B, and 5C show alternative configurations for sub-components of the XY-Stage and needle guidance template of FIGS. 1A and 1B in accordance with an illustrative embodiment.

FIGS. 6A and 6B show a method of operating the XY-Stage and needle guidance template of FIGS. 1A and 1B, respectively, in accordance with an illustrative embodiment.

### DETAILED DESCRIPTION

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [1] refers to the first reference in the list. All references cited and discussed in this specification are incorporated herein by reference in their entirety and to the same extent as if each reference were individually incorporated by reference.

An exemplary system and method are disclosed for a flexible XY-stage that spans a substantial portion of a subject's torso to assist in guiding a needle template or needle insertion apparatus for needle-based intervention procedures in an MRI scanner. The flexible XY-stage is optimally configured to span the entire torso of the subject in providing both (i) a large bendable frame that can be positioned over the subject and (ii) a low height profile, to optimally fit within the bore of an MRI scanner. The needle template or needle insertion apparatus is movable within a large movable space defined by the flexible XY-stage and guided by cables connected at the corners of the flexible XY-stage. The needle template or needle insertion apparatus has a plurality of mounting positions (e.g., rings) that allows a needle-based instrument to be mounted to the needle template or needle insertion apparatus and over a large degree of angle, notably 0° to 75° from vertical, allowing for more options for a clinician to place the needle-based instrument in the patient, e.g., to reach an abdomen target without damaging the obstacles (e.g., blood vessels).

The flexible XY-stage may be optimized by discretizing the curved surface into multiple flat surfaces and applying a planar kinematics model to solve for the robot kinematics. The exemplary system and method can provide precise position and orientation control of the needle because its domed needle template can, via customizable holes (i.e., rings), allow the needles to reach an abdomen target without damaging the obstacles (e.g., blood vessels). The exemplary system and method can notably conform to the patient's contours and maintain structure integrity. The flexible XY-stage can adapt to a broad range of patient types, body weight indices, and orientations. In some embodiments, the flexible XY-stage can incorporate a frame made at least in part of deformable materials (e.g., thermoplastic polyurethane (TPU)) or rigid materials with flexible structure design. In some embodiments, the XY-stage can employ motors to drive the needle guide at various locations and orientations. The XY-stage can also be controlled manually. While beneficial for a close-bore MRI scanner, the exemplary system and method can be employed for open-bore MRI, wide-bore MRI, and other MRI systems.

### Example System

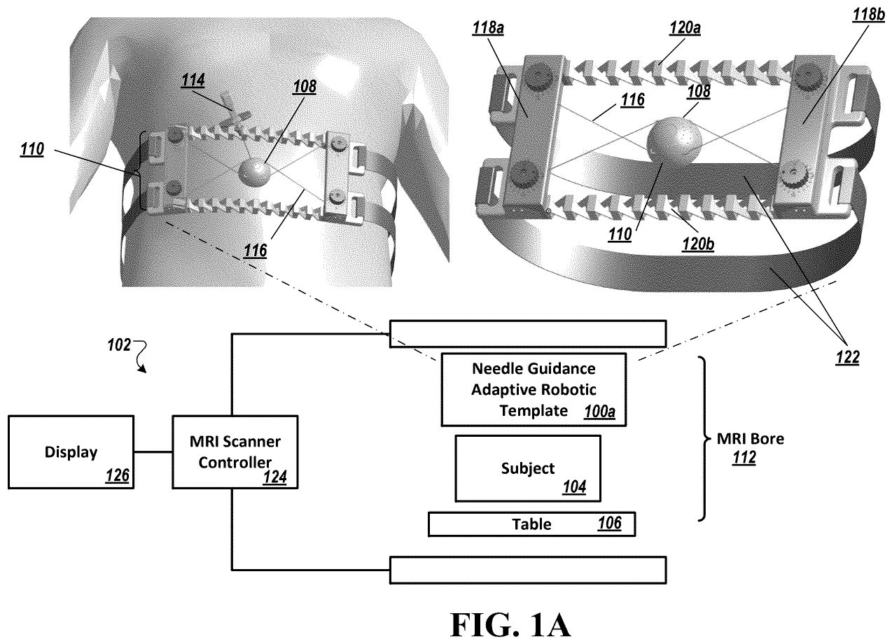

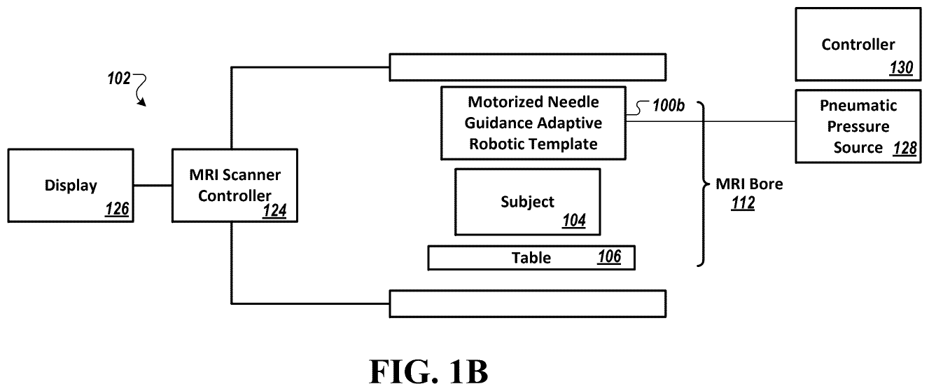

FIGS. 1A and 1B each shows an example XY-Stage and needle guidance template system 100 (shown as “Needle guidance adaptive robotic template” 100a and “Motorized Needle guidance adaptive robotic template” 100b, respectively) configured to operate with an MRI system 102 for needle guidance and/or placement in accordance with an illustrative embodiment. In the example shown in FIG. 1A, the exemplary XY-Stage and needle guidance template system 100 includes (i) a flexible XY-stage 110 configured to contour and securely position over the patient's body 104 and (ii) a domed needle template 108 configured with customizable holes 110 (i.e., rings) to allow the needle of a needle instrument 114 to reach an abdomen target without damaging any obstacles (e.g., blood vessels). The flexible XY-stage allows the domed needle template 108 to be guided and maintained at a position over the subject body 104 to which the needle of the needle instrument 114 is inserted into the subject's body 104. In maintaining the needle and needle instrument 114 at desired positions over the subject's body, the XY-Stage and needle guidance template system 100 can allow a clinician or surgeon to manually position the needle at the desired orientation and position more quickly as the subject 104 is moved in and out of the MRI bore 112 during re-scanning. FIG. 1B shows a similar mechanism employed in a motorized configuration to position the needle and needle instrument 114 at the desired positions over the subject's body 104; the embodiment allows the subject 104 to be maintained in the bore 112 during the positioning and re-positioning without having to be removed from the bore 112, though at a tradeoff of more device complexity and slightly more limited availability of guidance angle. A limiting constraint of the application is the limited space of the MRI bore 112 and the size of the subject 104.

FIG. 1B shows the motorized needle guidance adaptive robotic template 100b additionally coupled to a pressure source 128 that is controlled, via a valve, by a needle guidance controller 130 configured to regulate pressure or flow to pneumatic motors in the motorized needle guidance adaptive robotic template 100b to adjust the cable 116 to direct movement/positioning of the domed needle template 108 in the flexible XY-stage 110 to a desired position.

The MRI system 102 includes a table 106, MRI scanner controller 124, and display 126, among others. The subject 104 is situated on the table 106 located in the MRI scanner bore 112. The MRI scanner controller 124 is configured to acquire an MRI scan to allow visualization, via the display 126, of the internal body organ or tissue during placement of the needle instrument location or needle depth. In alternative embodiments, the MRI scanner controller 124 is configured to allow scanning of the subject, the scan being used to direct placement of the domed needle template 108 within the flexible XY-stage 110.

### Flexible and Expandable XY-Stage Frame for Needle Positioning

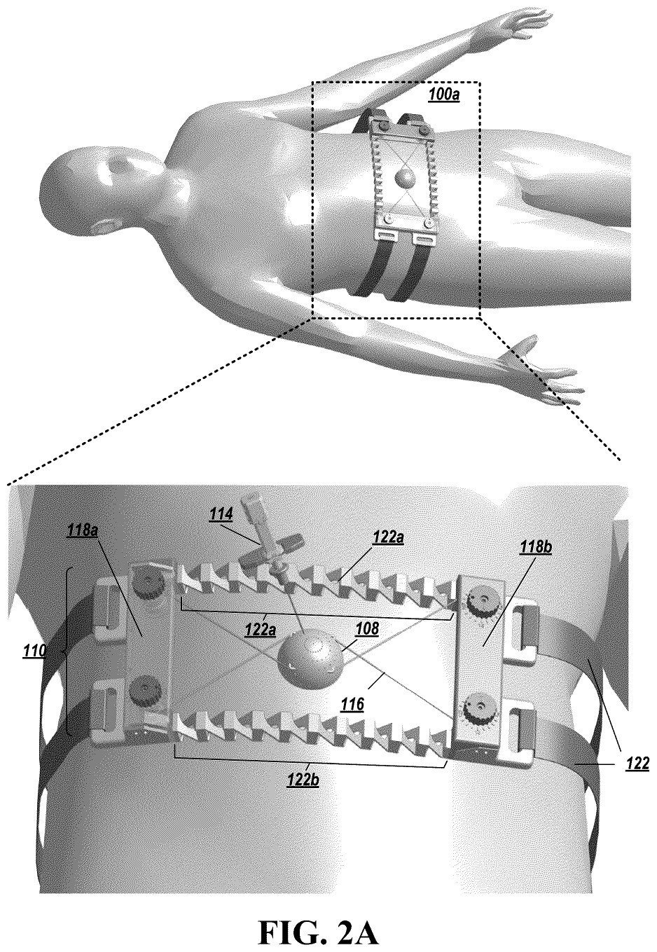

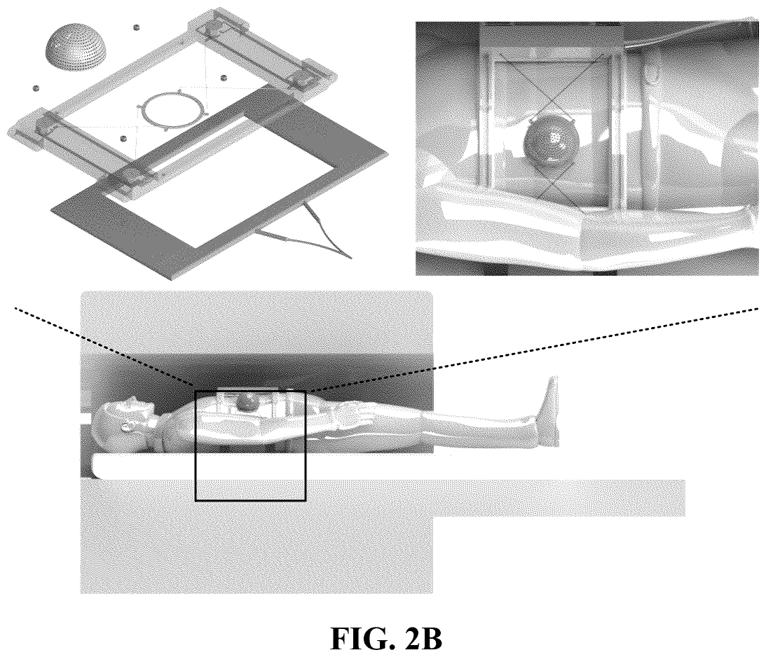

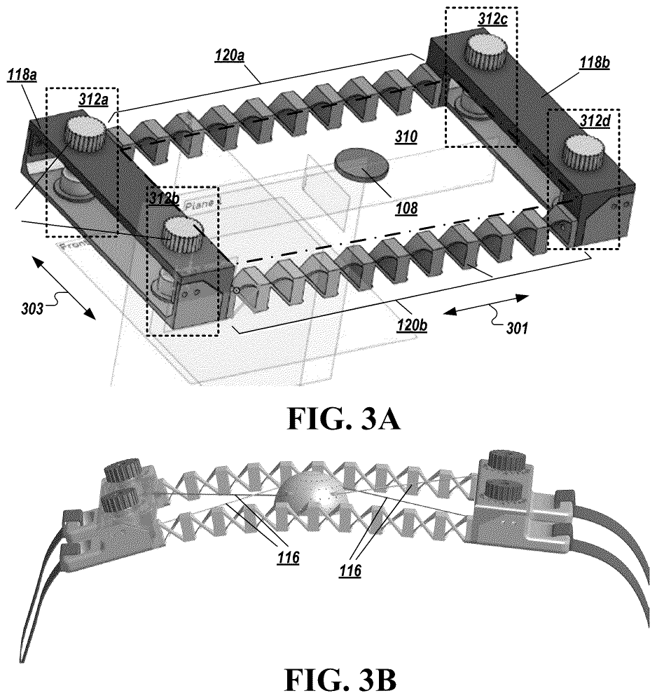

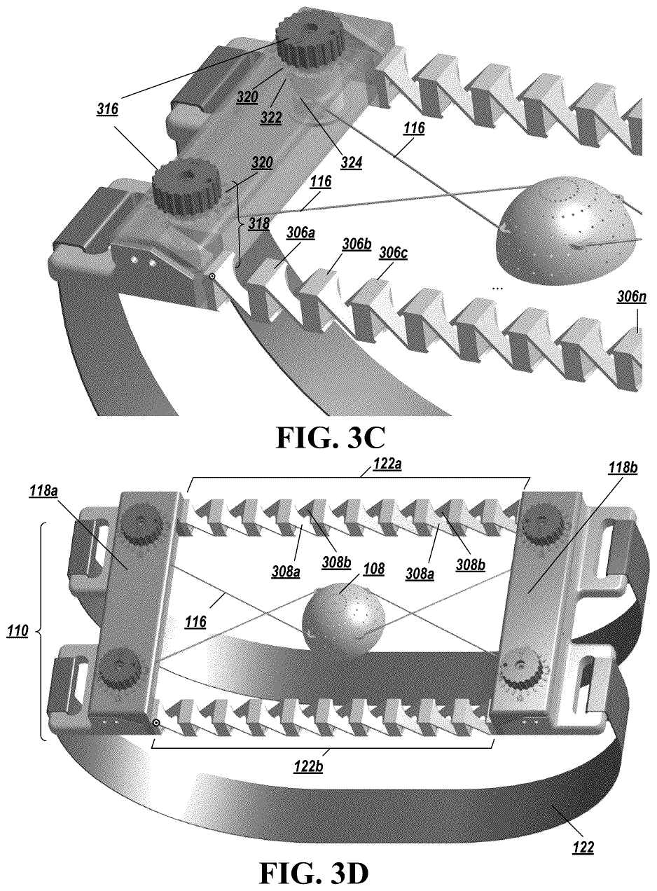

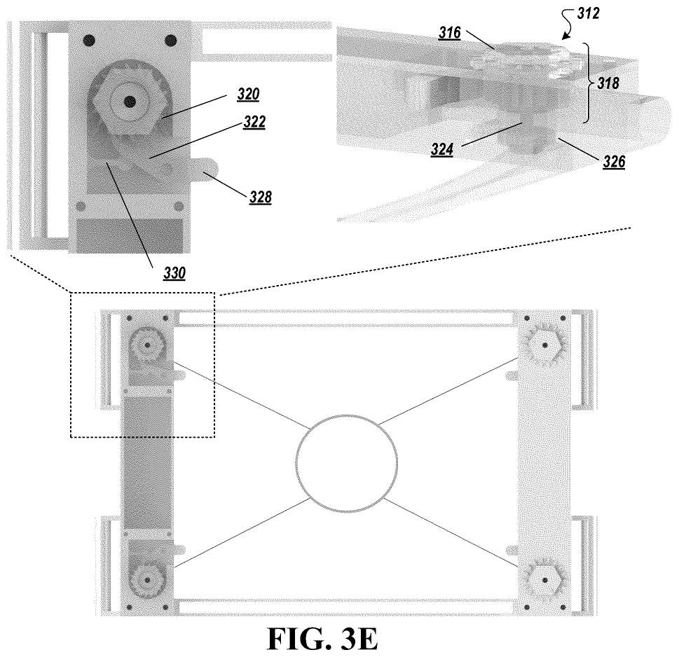

In FIGS. 2A, 2B, 3A, 3B, 4A, and 4B, the flexible XY-stage 110 is shown configured as a lightweight, rectangular-framed XY-stage configured to conform to the patient's contours while maintaining structural integrity, e.g., a stage for needle positioning. FIG. 2A shows the flexible XY-stage 110 positioned at a first position on the body. FIG. 2B shows the flexible XY-stage 110 positioned at another position on the body, notably on the side of the subject. Notably, the FIGS. 2A and 2B show flexible XY-stage 110 providing a large surface for guidance of the needle and flexible application to different body regions. The flexible XY-stage 110 includes mounting connections for straps 122 to circumscribe the body of the subject to secure the flexible XY-stage 110 to the subject. In FIG. 3B, the steady state position is shown where the flexible XY-stage 110 has an initial bend position; the steady state position is controlled by the tension/length of the cable 116. In a non-tensioned state, the flexible XY-stage 110 may rest in a flat/planar configuration.

The flexible XY-stage 110 may be fabricated from TPU, e.g., via precision additive manufacturing (e.g., InkBit). Other manufacturing techniques, additive or conventional, may be used. The size of the flexible XY-stage 110 is generally longer along the traversal direction (301) to provide a wide flexible frame that can provide greater needle insertion guide access along the frontal or back plane of the body. Different from rigid systems, the flexible XY-stage 110 show in FIG. 3A is configured to elongate to allow for different patient sizes and circumferences. The flexible frame configuration may be adapted/designed for a broad range of patient types and orientations, to provide expanded usable workspace and clinical flexibility. Notably, the flexible contouring mechanical design allows a larger/wider flexible XY-stage to be implemented while still fitting in the MRI bore 112 along with the patient, to allow for a larger area of coverage by the device.

While longer in the traversal direction, in FIG. 3A, the flexible XY-stage 110 is designed to be shorter in the sagittal direction 303 to allow more stable positioning along the body, e.g., at the torso, abdomen, or pelvic region. While the body is generally cylindrical, the top part of the cylinder can be larger than the bottom part (over the chest or abdomen region). The width and length of the flexible XY-stage 110 (e.g., ratio between the flexible arms 120 and the frame arm 118) may be a 1.5:1 ratio, a 2:1 ratio, a 3:1 ratio, or the like. It is contemplated that any ratio may be employed, though practically the flexible arms 120 should be longer than the rigid frame arms 118. In FIG. 3A, the ratio is about 2:1. The frame topology 108 may be designed depending on the patient's anatomy.

As shown in FIG. 3A, the flexible XY-stage 110 includes a first frame arm 118a and a second frame arm 118b connected by a set of flexible arms 120a, 120b to form an open flexible frame configured to bend around a body region, e.g., at the torso, abdomen, pelvic, or lower extremity region of a subject. The open flexible frame defines a movable space 310 for the domed needle template 108 (also referred to as the “movable assembly” 108) as a guiding component for a needle instrument. The flexible arms 120a, 120b are preferably bias to bend along a single axis/direction while being allowed to elongate to allow the flexible XY-stage 110 to bend and fit around the contour of the body section and adjusted to body sizes. In some embodiments, a portion of the flexible arms 120a, 120b may allow for rotation in a second direction, e.g., perpendicular to the primary direction, to allow for additional adjustments, e.g., to accommodate differences in anatomy. The rotation beneficially avoids an overt bending on the frame in a second direction. FIGS. 3A and 3B show different views of the flexible arms 120a, 120b. The flexible arms 120a, 120b are formed of a set of interconnected members having a plurality of nodes 306 (shown as 306a, 306b, 306c, . . . 306n; see FIG. 3B, top image) each connected to a nearby node 306 via two connecting members 308a, 308b (see FIG. 3B, bottom image). Each connecting member 308a, 308b is connected to each node at opposing sides to form a contra-opposing truss (shown as “X”). Each pair of connecting members 308a, 308b forms a bendable truss that allows the flexible arms 120a, 120b to bend in a single or generally single direction. In FIGS. 3A and 3B, the flexible arms 120a, 120b are formed of a single unitary structure, e.g., as a 3D printed part, made of a deformable material (e.g., TPU). In other configurations, the single unitary structure may be made of rigid plastic, metal having the nodes coupled by hinges or other bending mechanisms.

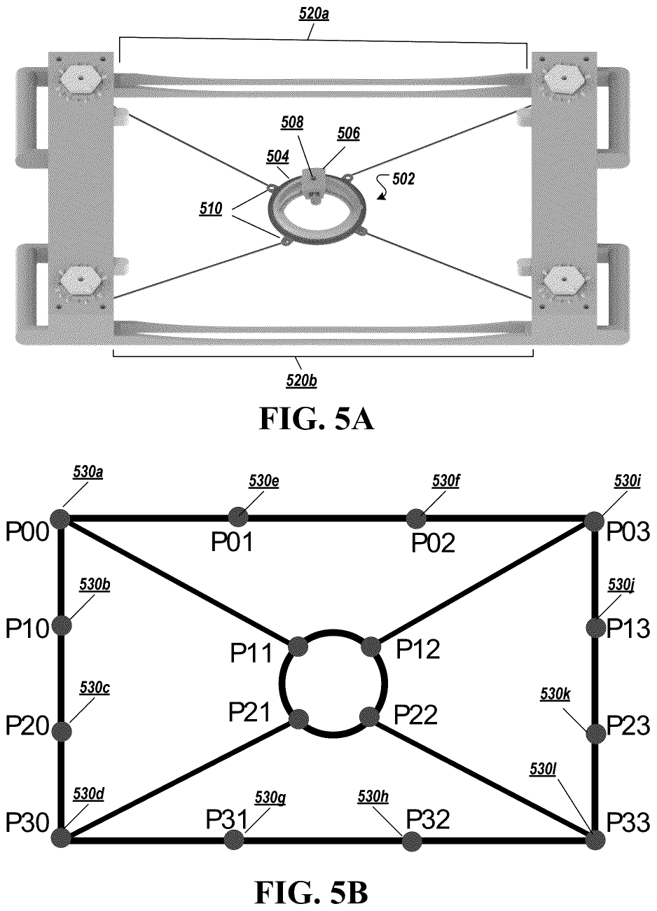

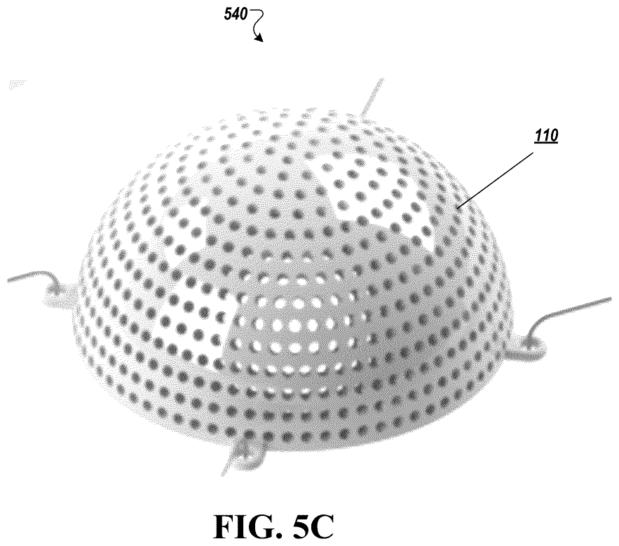

FIG. 5A shows the flexible arms 120a, 120b (shown as 502a, 502b) configured with a set of flexible beams. The beams are made out of a flexible material and are a shape such that bending is allowed along the length of the beam, but not along the width of the beam.

### Domed Needle Template for Orientation Control

FIGS. 3B, 3C, and 3D show an example of the domed needle template 108 operatively coupled to a flexible XY-stage 110, in accordance with an illustrative embodiment. FIG. 5A shows another needle insertion assembly 502 as an alternative for the domed needle template 108; the needle insertion assembly 502 is operatively coupled to a motorized XY-stage, in accordance with an illustrative embodiment.

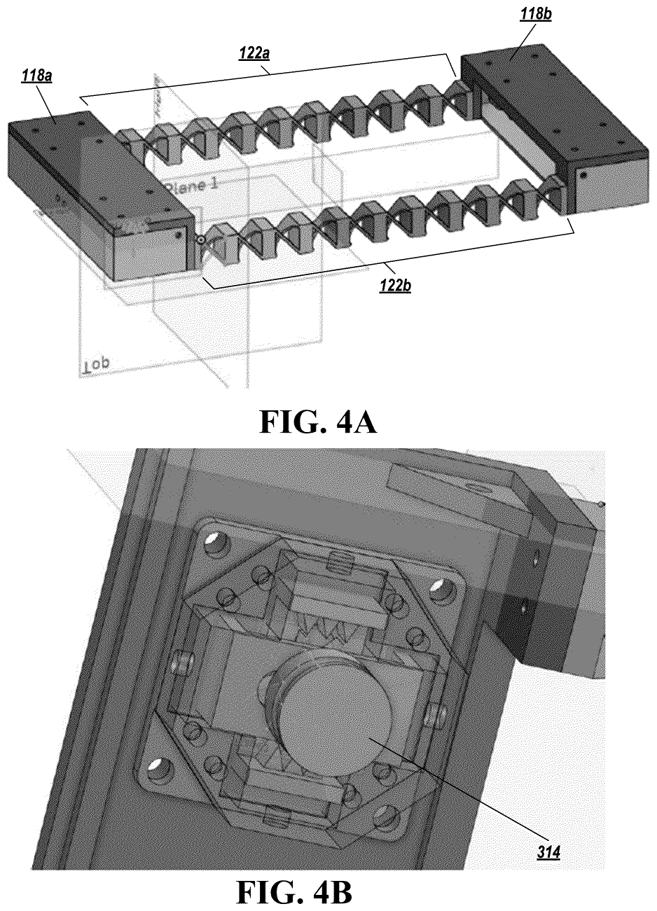

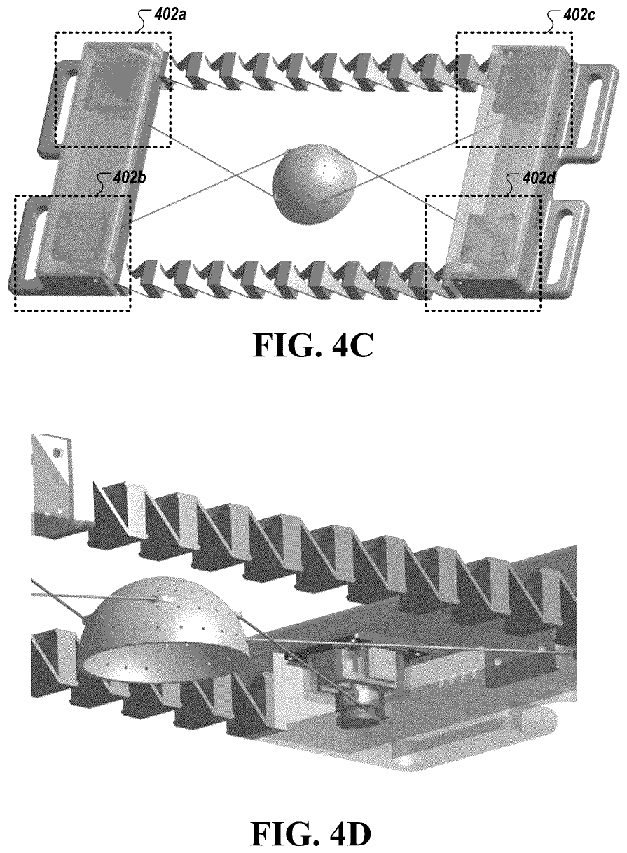

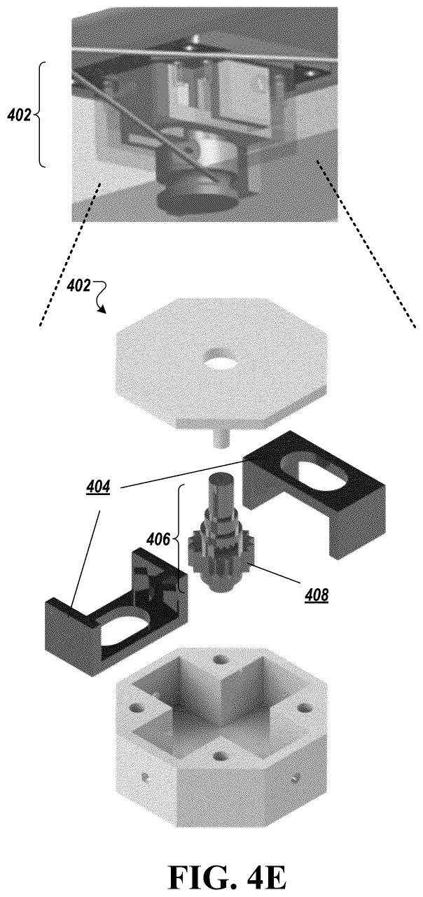

In FIG. 3B, the needle template 108 (referred to as the “movable assembly” 108) is configured to move within the movable space 310 defined within the open flexible frame. As shown in FIG. 3A, the flexible arms 120a, 120b each include a drive assembly 312 (shown as 312a, 312b, 312c, 312d) that includes a bearing ring 314 (see FIG. 4A) to guide the cable 116 operatively couple thereto. The drive assembly 312a-312d, e.g., comprises a ratchet and pawl subassembly fixably coupled to the bearing ring 314 along to shaft, the ratchet and pawl subassembly configured to provide resistance to or anchoring of the bearing ring 314 to guide/control the movement of the cable 116 to maintain the movable assembly 108 at a desired position. The cable 116 is preferably a high-strength, durable cable configured not to elongate under tension. The cable 116 may be connected to the drive assembly 312a-312d at, at least, 3 locations, to provided stability. The cable 116 may be arranged to connected to the connect to the needle template 108 in a diagonal form that overlaps with a paired nearby cable (e.g., see FIG. 3B, 3C, 3D). The crossing of the cables allows for rotation of the center ring when in the crossed configuration. The cable 116 can be alternatively arranged to connect to the needle template 108 in a non-diagonal form.

For orientation, the domed template 108 includes a plurality of customizable holes 110 to allow placement of a needle instrument 114. The domed template 108 is curved to so each hole 110 provides a different angle/orientation for the insertion of the instrument 114. The domed template 108 (span half a hemisphere) can provide a wide range of insertion angles, allowing for clinically steeper insertions, up to ±75° from the vertical (e.g., 0° to ±75°). In some embodiments, the insertion angle can be greater than 75°. The insertion improves upon other systems, e.g., having insertions up to ±40° from the vertical. The wider range may reduce some orientation precision, but would be more relevant for abdominal needle interventions where insertion angles may vary. The template 108 may rest on the patient's body (e.g., resting on the skin or clothing). The bearing ring can decrease the friction during the movement of the cable to provide smooth translation of the cables via the XY-stage. In FIG. 3B, the domed template 108 provides a placement at 0°, ±15°, ±30°, ±45°, ±60°, and 75° for the full 360 degrees. Other angles may be provided in the noted range. The domed template 108 may operate with the cable configuration (e.g., in a cross configuration) to provide a full 360 degrees of placement access with granular control. Other configuration of the dome may be employed (e.g., see FIG. 5C).

### Manual Operation of the Drive Assembly

As discussed above, in FIG. 3A, the drive assembly 312 (shown as 312a, 312b, 312c, 312d) includes a bearing ring 314 (see FIG. 4A) to guide the cable 116 operatively couple thereto. FIG. 3E shows an internal view of the flexible XY-stage 110 and the drive assembly 312a-312d. In FIG. 3E, the drive assembly 312a-312d is shown comprising a ratchet and pawl subassembly 318 fixably coupled to the bearing ring 314 along a shaft 324, the ratchet and pawl subassembly 318 configured to provide resistance to or anchoring of the bearing ring 314 to guide/control the movement of the cable 116 to maintain the movable assembly 108 at a desired position. The drive assembly 312a-312d includes a knob 316 (FIG. 3C) to allow manual rotation of the ratchet and pawl assembly 318. The rachet and pawl assembly 318 includes a rachet 320 and pawl 322 to adjust the tension of the cable 116. Each drive assembly 312a-312d includes a shaft 324 coupled to the rachet and pawl assembly 318 and knob 316, the shaft 324 having a ring guide 314 to guide the cable 116. The drive assembly 312a-312d is configured to release the tension of the cable 116 by an actuation of the knob 328 coupled to the pawl 322. The pawl 328 is connected via a spring or resilient member 330 to bias the pawl 322 against the rachet 320. The pawl 322 may allow for rotation in one direction (default being to tighten the cable) and may be actuated via the knob 328 to allow rotation in the other direction (loosen). The drive assembly 312a-312d may lock by the release of the knob 322.

The cable 116 may route individually to each of the drive assembly 312; the system has 4 cables. In other embodiments, a cable 116 may route to two of the drive assembly 312; the system has 2 cables. In other embodiments, a single cable 116 may route to four drive assembly 312; the system has a single cable.

### Motorized Operation of the Drive Assembly

The exemplary system can extend the current cable-driven flat case kinematics to the curved surface scenario using the constant curvature assumption or by discretizing the curved surface into many flat surfaces, then integrating each flat surface's kinematics for the comprehensive model. The exemplary system can also incorporate the task-space closed-loop control frame to address modeling uncertainties. In the example shown in FIG. 4A, the flexible XY-stage 110 is configured with a a pneumatic motor pair configured to drive the cable to provide a core-XY-like mechanism, to allow needle alignment across the entire frame.

In FIGS. 4A-4E, the flexible XY-stage 110 is also shown configured as a lightweight, rectangular-framed XY-stage configured to conform to the patient's contours while maintaining structural integrity, e.g., a stage for needle positioning. The flexible XY-stage 110 may be fabricated from TPU or other manufacturing techniques, additive or conventional. The size of the flexible XY-stage 110 is generally longer along the traversal direction (e.g., 301) to provide a wide flexible frame that can provide greater needle insertion guide access along the frontal or back plane of the body. Notably, the flexible contouring mechanical design allows a larger/wider flexible XY-stage to be implemented while still fitting in the MRI bore 112 along with the patient, to allow for a larger area of coverage by the device.

As shown in FIG. 4A, the flexible XY-stage 110 includes a first frame arm 118a and a second frame arm 118b connected by a set of flexible arms 120a, 120b to form an open flexible frame configured to bend around a body region, e.g., at the torso, abdomen, pelvic, or lower extremity region of a subject. The open flexible frame defines a movable space (e.g., 310) for the movable assembly 108 as a guiding component for a needle instrument. The flexible arms 120a, 120b are bias to bend along a single axis/direction while being allowed to elongate to allow the flexible XY-stage 110 to bend and fit around the contour of the body section and be adjusted to body sizes.

In FIG. 4C, the movable assembly (108) is configured to move within the movable space 310 defined within the open flexible frame. The flexible arms 120a, 120b each include a motorized drive assembly 402 (shown as 402a, 402b, 402c, 402d) that includes a pneumatic motor to guide the cable 116 operatively couple thereto. The cable 116 is preferably a high-strength, durable cable configured not to elongate under tension. FIGS. 4C and 4D show additional views of the flexible XY-stage 110 and motorized drive assembly 402a-402d.

FIG. 4E shows an exploded view of the motorized drive assembly 402a-402d. In FIG. 4E, a motorized drive assembly 402 (e.g., 402a-404d) includes a pneumatic motor 404 (gears not shown) that is coupled to a shaft 406 having a corresponding gear 408 to engage with the gears of the motor 404. Pneumatic motor 404 is employed to allow actuation in a high magnetic field environment in the MRI bore. The pneumatic motor 404 is coupled to an air or vacuum source and a controllable valve. A controller configured to pneumatically actuate the motor 404 via the controllable valve. An example motorized needle insertion mechanism is described in PCT Patent Application No. WO/2023/137155, which is incorporated by reference herein.

### Additional Configurations

FIGS. 5A-5C show additional configurations for the XY-Stage and needle guidance template system 100.

Alternative Movable Assembly. In FIG. 5A, an alternative movable assembly 502 (previously shown as 108) is shown. The movable assembly 502 includes a ring assembly 504 configured to retain a rotating retaining member 506. The rotating retaining member 506 includes a mounting hole 508 for placement of a needle or needle instrument. The ring assembly 504 includes a plurality of fixed mounting points 510 for the cable 116 to connect.

Alternative Interconnecting Member. FIG. 5A also shows an alternative interconnecting member 520a, 520b (previously shown as 120a, 120b) as a flexible beam.

The beams are made out of a flexible material and are a shape such that bending is allowed along the length of the beam, but not along the width of the beam. As shown in FIG. 5A, the beam has a thicker, filled width that does not allow bending and and is relatively thinner along the length to allow for a natural bending of the beam along the length.

Shape Estimation for Frame. FIG. 5B shows points employed for the estimation of the shape of the frame. In FIG. 5B, the points 530a-5301 are shown as P00, P10, P20, P30, P01, P02, P31, P32, P03, P13, P23, and P33, respectively. The attachment points may be along any points along the lines of 530a-530d and 530i-5301. The number of mounting points may be 3, 4, 5, 6, 7, and 8. Alternative Domed Template. FIG. 5C shows an alterative domed template 108 (shown as 540) having high density of holes to allow greater number of placement options of a needle instrument 114. The domed template 540 is also curved to so each hole 110 provides a different angle/orientation for the insertion of the instrument 114. The domed template 540 (span half a hemisphere) can provide a wide range of insertion angles, allowing for clinically steeper insertions, up to ±75° from the vertical (e.g., 0° to ±75°) at 5° increments (e.g., 0°, 5°, 10°, 15°, 20°, 25°, 30°, 35°, 40°, 45°, 50°, 55°, 60°, 65°, 70°, 75°). In some embodiments, the insertion angle can be greater than 75°.

### Example Method

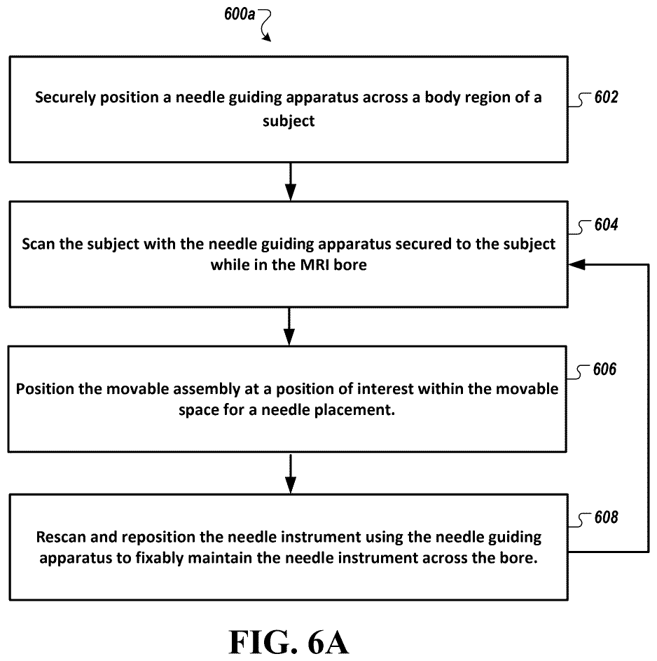

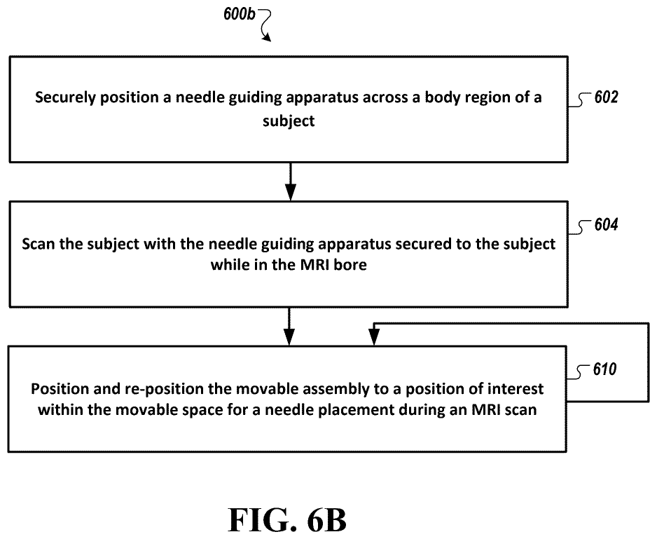

FIGS. 6A and 6B each show an example method 600a, 600b of operation for the exemplary XY-Stage and needle guidance template system 100 in accordance with an illustrative embodiment. As non-limiting examples, methods 600a, 600b may be used for radio-frequency ablation, microwave ablation, cryotherapy, biopsy, brachytherapy, drug delivery applications, among other procedures involving a needle-based instrument. Additionally, methods 600a, 600b may be used for the treatment and diagnosis of prostate cancer, gynecological cancer, and breast cancer, among others.

Method #1. Method 600a includes securely positioning (602) (e.g., via straps) a needle guiding apparatus (e.g., 100) across a body region of a subject, wherein the needle guiding apparatus (e.g., 100) comprises: (i) a first frame arm and a second frame arm (e.g., 118) connected by a set of interconnected members (e.g., 120, 520) to form an open flexible frame configured to bend (e.g., along a single axis) around a body region (e.g., torso, pelvic, or lower extremity region) of a subject, the open flexible frame defining a movable space for a guiding component; and (ii) a movable assembly (e.g., 108, 502) as the guiding component tethered, via a cable (e.g., 116), to each corner of the first arm and the second arm (e.g., 118), the open flexible frame (e.g., 110) maintaining the cable in tension to guide movement of the movable assembly in the movable space, the movable assembly including a guide or motorized component to fixably maintain a needle instrument for needle placement. The first frame arm and a second frame arm (e.g., 118) may include straps (e.g., 122) to secure the device (e.g., 100) to the subject.

Method 600a then includes scanning (604) the subject with the needle guiding apparatus (e.g., 100) secured to the subject while in the MRI bore.

Method 600a then includes positioning (606) the movable assembly at a position of interest within the movable space for a needle placement (e.g., for the needle instrument 114). The operation may include withdrawing the subject from the bore to position the movable assembly (e.g., 108, 502) at a position of interest.

Method 600a then includes rescanning and repositioning (608) the needle instrument (e.g., 114) using the needle guiding apparatus to fixably maintain the needle instrument across the bore. In maintaining the needle placement via operation 606, the needle instrument (e.g., 114) and needle may be guided to the organ or tissue of interest more quickly. The operation of 606 and 608 may be repeated until the needle is positioned at the desired location as well as for needle insertion.

Method #2. Method 600b includes securely positioning (602) (e.g., via straps) a needle guiding apparatus (e.g., 100b) across a body region of a subject, wherein the needle guiding apparatus (e.g., 100b) comprises: (i) a first frame arm and a second frame arm connected by a set of interconnected members to form an open flexible frame configured to bend (e.g., along a single axis) around a body region (e.g., torso, pelvic, or lower extremity region) of a subject, the open flexible frame (e.g., 110) defining a movable space for a guiding component; and (ii) a movable assembly (e.g. 108, 502) as the guiding component tethered, via a cable (e.g., 116), to each corner of the first arm and the second arm (e.g., 118), the open flexible frame maintaining the cable (e.g., 116) in tension to guide movement of the movable assembly (e.g., 108, 502) in the movable space, the movable assembly (e.g., 108, 502) including a guide or motorized component to fixably maintain a needle instrument for needle placement, wherein the movable assembly and/or the open flexible frame is motorized.

Method 600b includes scanning (604) the subject with the needle guiding apparatus (e.g., 100b) secured to the subject while in the MRI bore.

Method 600b includes positioning and repositioning (610) the movable assembly at a position of interest within the movable space for a needle placement during an MRI scan.

Method 600b may additionally include inserting, via the motorized movable assembly, the needle instrument into the subject while the subject is being scanned.

In Method 600a, 600b, the open flexible frame may define the movable space for a substantial portion of a torso region as the body region of the subject (e.g., greater than 50% of torso area). The open flexible frame may span across a plane when in an unbending configuration, wherein the movable assembly is positionable and movable in the plane to provide a low-profile height device. The open flexible frame is configured to span a substantial portion of an MRI bore (e.g., at least 50% along the largest plane defined in the bore).

In some embodiments, the movable assembly (e.g., domed template) may include as a substrate having a plurality of guiding holes for placement of the needle instrument, including a first guiding hole and a second guiding hole, wherein a first guiding hole provides a first orientation of the needle instrument, and wherein a second guiding hole provides a second orientation of the needle instrument, the guiding holes providing an insertion angles range of at least 60 degrees (e.g., 0° to 85° from vertical or normal of the body surface).

In some embodiments, the movable assembly includes a substrate having a plurality of guiding holes for placement of the needle instrument. The movable assembly may have a half-sphere profile.

In some embodiments, the open flexible frame has a symmetric rectangular or square profile. In alternative embodiments, the open flexible frame has an asymmetric rectangular or square profile. In either embodiment, the open flexible frame is configured to conform to a patient's contours at the body region.

In some embodiments, at least the set of interconnected members is made of a deformable material (e.g., thermoplastic polyurethane).

In some embodiments, the first frame arm and a second frame arm may include a drive assembly comprising at least one bearing ring to guide the cable operatively couple thereto, the drive assembly (e.g., a ratchet and pawl subassembly) configured to provide resistance to or anchoring of the movement of the cable to maintain the movable assembly at a position. In some embodiments, two or more cables are employed to traverse across the four corners of the open flexible frame to connect to the movable assembly. In some embodiments, a single cable is employed to traverse across the four corners of the open flexible frame to connect to the movable assembly.

In some embodiments, the drive assembly includes at least one actuator fixably coupled to the at least one bearing ring, the actuator being configured to drive the at least one bearing ring to reduce friction between the movable assembly and the patient's skin.

### DISCUSSION

The challenges of current rigid, body-mounted robotic designs have limited the development of magnetic resonance-guided (MR-guided) robots for abdominal needle interventions. These limitations include: (1) Constrained Reachable Workspace: to ensure a compact footprint that fits comfortably on the patient, the workspace of rigid robots is limited, which restricts needle positioning options and the available angles for insertion; (2) Increased Height and Patient Restrictions: adding stages to achieve 4-degree-of-freedom (4-DoF) control for needle position and orientation increases the robot's height, excluding certain patient demographics and bore sizes from use; (3) Mounting Limitations: current body-mounted systems require parallel mounting over the patient, precluding lateral insertions, which may be clinically optimal in some cases; and (4) Complexity and Sterilization Challenges: rigid, intricate robotic designs are costly and difficult to sterilize, limiting practical clinical deployment.

To address these challenges, the exemplary system and method are developed as a solution for MR-guided robotic needle positioning. The exemplary system includes (i) a flexible XY-stage that contours to the patient's body and (ii) a domed needle template that enables enhanced orientation control.

The exemplary system includes a lightweight frame, as its XY-stage, configured to conform to the patient's contours while maintaining structural integrity. The adaptivity may be achieved by fabricating the frame with deformable materials such as TPU or rigid materials, but with a flexible structure design. This may allow the exemplary system to adapt to various patient types, BWI, and orientation. 4 motors may be used to drive the system to allow the customized needle guide to move at different locations and orientations.

The current template may only provide the position control. Different from the current template, the domed needle insertion template in the exemplary system may achieve position and orientation control by having multiple customizable holes. The wide range of orientation control may allow clinicians to reach a target without damaging the obstacles, such as blood vessels.

The exemplary system may provide needle-based interventions inside the abdomen (e.g., liver, kidney, pancreas, stomach, etc.). The exemplary system can also provide efficient radio-frequency ablation, microwave ablation, cryotherapy, biopsy, brachytherapy, and drug delivery applications. Additionally, the dome needle insertion template of the exemplary system may be used for the treatment and diagnosis of prostate cancer, gynecological cancer, and breast cancer.

Additionally, in the current systems, the needles are inserted with no or limited guidance (via a planar guiding template). This procedure requires extensive training, and the treatment outcome depends on the clinician's skill. A guiding system that can guide the needle insertion position and orientation is lacking.

### CONCLUSION

The construction and arrangement of the systems and methods, as shown in the various implementations, are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementation of the present disclosure may be implemented using existing computer processors, or by a special-purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media accessed by a general-purpose or special-purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data that cause a general-purpose computer, special-purpose computer, or special-purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on the designer's choice. All such variations are within the scope of the disclosure. Likewise, software implementations could be accomplished with standard programming techniques with rule-based logic and other logic to accomplish the various connection steps, processing steps, comparison steps, and decision steps.

Each and every feature described herein, and each and every combination of two or more of such features, is included within the scope of the present invention, provided that the features included in such a combination are not mutually inconsistent.

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

While the methods and systems have been described in connection with certain embodiments and specific examples, it is not intended that the scope be limited to the particular embodiments set forth, as the embodiments herein are intended in all respects to be illustrative rather than restrictive.

The following patents, applications, and publications, as listed below and throughout this document, are hereby incorporated by reference in their entirety herein.

What is claimed:

1. An apparatus comprising:
a first frame arm and a second frame arm connected by a set of flexible arms to form an open flexible frame configured to bend around a body region of a subject, the open flexible frame defining a movable space for a guiding component; and
a movable assembly as the guiding component tethered, via a cable, to each corner of the first arm and the second arm, the open flexible frame maintaining the cable in tension to guide movement of the movable assembly in the movable space, the movable assembly including a guide or motorized component to fixably maintain a needle instrument for needle placement.

2. The apparatus of claim 1, wherein the open flexible frame defines the movable space for a substantial portion of a torso region as the body region of the subject.

3. The apparatus of claim 1, wherein the open flexible frame spans across a plane when in an unbending configuration, wherein the movable assembly is positionable and movable in the plane to provide a low-profile height device.

4. The apparatus of claim 1, wherein the open flexible frame is configured to span a substantial portion of an MRI bore.

5. The apparatus of claim 1, wherein the movable assembly includes as a substrate having a plurality of guiding holes for placement of the needle instrument, including a first guiding hole and a second guiding hole, wherein a first guiding hole provides a first orientation of the needle instrument, and wherein a second guiding hole provides a second orientation of the needle instrument, the guiding holes providing an insertion angles range of at least 60 degrees.

6. The apparatus of claim 1, wherein the movable assembly includes a substrate having a plurality of guiding holes for placement of the needle instrument.

7. The apparatus of claim 1, wherein the open flexible frame has a symmetric rectangular or square profile.

8. The apparatus of claim 1, wherein the open flexible frame has an asymmetric rectangular or square profile.

9. The apparatus of claim 1, wherein the open flexible frame is configured to conform to a patient's contours at the body region.

10. The apparatus of claim 1, wherein at least the set of interconnected members is made of a deformable material.

11. The apparatus of claim 1, wherein the first frame arm and a second frame arm comprise:
a drive assembly comprising at least one bearing ring to guide the cable operatively couple thereto, the drive assembly configured to provide resistance to or anchoring of the movement of the cable to maintain the movable assembly at a position.

12. The apparatus of claim 11, wherein the drive assembly includes at least one actuator fixably coupled to the at least one bearing ring, the actuator being configured to drive the at least one bearing ring to reduce friction between the movable assembly and the patient's skin.

13. The apparatus of claim 1, wherein the movable assembly has a half-sphere profile.

14. The apparatus of claim 1, wherein the first frame arm and a second frame arm include straps.

15. The apparatus of claim 11, wherein the drive assembly comprises a ratchet and pawl subassembly to provide resistance to or anchoring of the movement of the cable.

16. The apparatus of claim 1, wherein two or more cables are employed to traverse across the four corners of the open flexible frame to connect to the movable assembly.

17. The apparatus of claim 1, wherein a single cable is employed to traverse across the four corners of the open flexible frame to connect to the movable assembly.

18. A method comprising:
securely positioning a needle guiding apparatus across a body region of a subject, wherein the needle guiding apparatus comprises:
(i) a first frame arm and a second frame arm connected by a set of interconnected members to form an open flexible frame configured to bend around a body region of a subject, the open flexible frame defining a movable space for a guiding component; and
(ii) a movable assembly as the guiding component tethered, via a cable, to each corner of the first arm and the second arm, the open flexible frame maintaining the cable in tension to guide movement of the movable assembly in the movable space, the movable assembly including a guide or motorized component to fixably maintain a needle instrument for needle placement;
scanning the subject with the needle guiding apparatus secured to the subject while in the MRI bore;
positioning the movable assembly at a position of interest within the movable space for a needle placement;
rescanning and repositioning the needle instrument using the needle guiding apparatus to fixably maintain the needle instrument across the bore.

19. A method comprising:
securely positioning a needle guiding apparatus across a body region of a subject, wherein the needle guiding apparatus comprises:
(i) a first frame arm and a second frame arm connected by a set of interconnected members to form an open flexible frame configured to bend around a body region of a subject, the open flexible frame defining a movable space for a guiding component; and
(ii) a movable assembly as the guiding component tethered, via a cable, to each corner of the first arm and the second arm, the open flexible frame maintaining the cable in tension to guide movement of the movable assembly in the movable space, the movable assembly including a guide or motorized component to fixably maintain a needle instrument for needle placement, wherein the movable assembly and/or the open flexible frame is motorized;
scanning the subject with the needle guiding apparatus secured to the subject while in the MRI bore; and
positioning and repositioning the movable assembly at a position of interest within the movable space for a needle placement during an MRI scan.

20. The method of claim 19, further comprising:
inserting, via the motorized movable assembly, the needle instrument into the subject while the subject is being scanned.
